(19) United States
(12) Patent Application Publication
(10) Pub. No.: US 2005/0101608 A1
(43) Pub. Date: May 12, 2005

(54) Iloprost in combination therapies for the treatment of pulmonary arterial hypertension

(76) Inventor: Donald J. Santel, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/945,255

(22) Filed: Sep. 20, 2004

Related U.S. Application Data
(60) Provisional application No. 60/505,653, filed on Sep. 24, 2003.

(51) Int. Cl. A61K 31/519; A61K 31/41; A61K 31/557

(52) U.S. Cl. 514/252.16; 514/262.1; 514/381; 514/573

(57) ABSTRACT

Preferred embodiments of the present invention are related to novel therapeutic drug combinations and methods for treating pulmonary arterial hypertension. More particularly, aspects of the present invention are related to using a combination of iloprost and at least one additional agent, selected from the group consisting of an endothelin receptor antagonist and a PDE inhibitor.

### RELATED APPLICATIONS

This application claims the benefit of U.S. Provisional Application No. 60/505,653 filed Sep. 24, 2003, the entire disclosure of which is hereby expressly incorporated by reference in its entirety.

### FIELD OF THE INVENTION

Embodiments of this invention are related to using iloprost in combination with one or more additional agents, preferably an endothelin receptor antagonist and/or a PDE inhibitor, for treating and/or preventing pulmonary arterial hypertension.

### BACKGROUND OF THE INVENTION

Pulmonary arterial hypertension is a debilitating disease characterized by an increase in pulmonary vascular resistance leading to right ventricular failure and death. Pulmonary arterial hypertension (PAH) with no apparent cause is termed primary pulmonary hypertension (PPH). Recently, various pathophysiological changes associated with this disorder, including vasoconstriction, vascular remodeling (i.e. proliferation of both media and intima of the pulmonary resistance vessels), and in situ thrombosis have been characterized (e.g., D'Alonzo, G. E. et al. 1991 Ann Intern Med 115:343-349; Palevsky, H. I. et al. 1989 Circulation 80:1207-1221; Rubin, L. J. 1997 N Engl J Med 336:111-117; Wagenvoort, C. A. & Wagenvoort, N. 1970 Circulation 42:1163-1184; Wood, P. 1958 Br Heart J20:557-570). Impairment of vascular and endothelial homeostasis is evidenced from a reduced synthesis of prostacyclin (PGI₂), increased thromboxane production, decreased formation of nitric oxide and increased synthesis of endothelin-1 (Giaid, A. & Saleh, D. 1995 N Engl J Med 333:214-221; Xue, C & Johns, R. A. 1995 N Engl J Med 333:1642-1644). The intracellular free calcium concentration of vascular smooth muscle cells of pulmonary arteries in PPH has been reported to be elevated.

Current therapies for pulmonary hypertension are unsatisfactory. These typically involve calcium cannel antagonists, prostacyclins, endothelin receptor antagonists and long-term anticoagulant therapy. However, each treatment has limitations and side effects.

Consequently there is a long felt need for a new and combined medicament for the treatment of PAH, preferably employing lower doses of the active agents, which exhibits fewer or no adverse effects (i.e., less toxicity) and a favorable profile in terms effectiveness in patients in different stages of PAH.

### SUMMARY OF THE INVENTION

A therapeutic combination for the treatment of PAH is disclosed in accordance with an embodiment of the present invention. The therapeutic combination preferably comprises a prostacyclin and at least one additional agent, selected from the group consisting of an endothelin receptor antagonist, a PDE inhibitor, and a calcium channel blocker, wherein the prostacyclin and the at least one additional agent are provided at dosages sufficient to ameliorate at least one symptom associated with PAH.

In one embodiment, the prostacyclin is selected from the group consisting of iloprost, treprostinol, and beraprost. In another embodiment, the endothelin receptor antagonist is selected from the group consisting of bosentan, sitaxentan, and ambrisentan. In another embodiment, the prostacyclin is iloprost and the at least one additional agent is bosentan. In another embodiment, the iloprost is aerosolized. In another embodiment, the at least one additional agent comprises a PDE inhibitor selected from the group consisting of sildenafil (Viagra®), tadalafil (Cialis®) and vardenafil (LEVITRA®).

A method of treating PAH is disclosed. The method comprises administering effective amounts of a therapeutic combination comprising a prostacyclin and at least one additional agent, selected from the group consisting of an endothelin receptor antagonist, a PDE inhibitor, and a calcium channel blocker. In one preferred embodiment, the method comprises administering iloprost in combination with at least one additional agent selected from the group consisting of bosentan, sitaxentan, ambrisentan, sildenafil, tadalafil and vardenafil.

### DETAILED DESCRIPTION OF THE PREFERRED EMBODIMENT

In an embodiment of the present invention, a combination therapy is disclosed for treating pulmonary arterial hypertension. In one embodiment, the combination therapy involves administering an effective amount of a prostacyclin (PGI₂, stable analogs thereof (e.g., iloprost), or inducers thereof (e.g., cicletanine)), preferably an epoprostenol analog, and most preferably iloprost, in combination with one or more additional agents. The one or more additional agents may be administered together with the prostacyclin, for example in a single tablet or capsule, or the additional agents may be administered separately from the prostacyclin. In one preferred embodiment, the prostacyclin is aerosolized. In one embodiment, a second agent is employed, such as an endothelin receptor antagonist, that modulates the vasostate (e.g., vasodilation) of blood vessels through a mechanism which is distinct from that of iloprost. Preferably, the endothelin receptor antagonist is selected from the group consisting of bosentan (Tracleer™, Actelion), ambrisentan (Myogen) and sitaxentan (Encysive Pharmaceuticals). In another embodiment, a second agent is employed that modulates prostacyclin activity, bioavailability, half-life, or ameliorates an undesirable side-effect of the prostacyclin. In one preferred embodiment, the second agent is a PDE inhibitor adapted to enhance the prostacyclin activity, preferably selected from the group consisting of enoximone, milrinone (Primacor®), Anrinone (Inocor®), sildenafil (Viagra®), tadalafil (Cialis®) and vardenafil (LEVITRA®).

Epoprostenol Derivatives

A continuous infusion of prostacyclin (Flolan®, GlaxoSmithKline) was the first therapy shown to reduce mortality in a controlled study of patients with severe pulmonary hypertension. However, its use is associated with a number of serious drawbacks (Barst R. J. et al. 1996 N Engl J Med 334:296-301; Badesch D. B. et al. 2000 Ann Intern Med 132:425-434). The lack of pulmonary selectivity results in systemic side effects, tolerance leads to progressive increases in the dose, and there may be recurrent infections of the intravenous catheter. As an alternative, inhaled nitric oxide possesses pulmonary selectivity, but it is less potent than prostacyclin in the pulmonary vasculature. Moreover, an interruption in the inhalation of continuous nitric oxide may cause rebound pulmonary hypertension. Designed to combine the beneficial effects of prostacyclin with those of an inhalational application, aerosolized prostacyclin was found to be a potent pulmonary vasodilator in patients with acute respiratory failure, exerting preferential vasodilatation in well-ventilated lung regions (Walmrath D. et al. 1993 Lancet 342:961-962; Walmrath D. et al. 1995 Am J Respir Crit Care Med 151:724-730; Walrnrath D. et al. 1996 Am J Respir Crit Care Med 153:991-996; Zwissler B. et al. 1996 Am J Respir Crit Care Med 154:1671-1677). Similar results were obtained in spontaneously breathing patients who had lung fibrosis and severe pulmonary hypertension (Olschewski H. et al. 1999 Am J Respir Crit Care Med 160:600-607).

Three epoprostenol analogs have been studied in the treatment of PAH: treprostinil (Remodulin®, United Therapeutics), beraprost, and iloprost. Treprostinol is a stable analogue of epoprostenol, which is given continuously subcutaneously. Escalation of dosage has been limited by significant infusion site pain. Thus many patients do not receive therapeutic doses. Beraprost is active orally and has shown a benefit in a study in PAH at 3 and 6 months but not at 9 or 12 months (Barst, R J, J Am Coll Cardiol, 2003. June 18;41(12):2119-25. Iloprost can be given intravenously or by nebulizer. The advantages of the nebulizer method of delivery is that less of the substance reaches the systemic circulation (a “pseudoselective” pulmonary vasodilator). Iloprost is generally given six to nine times a day, which may disrupt the patient's lifestyle; dosing frequency may be reduced by combining iloprost with an agent having a therapeutic effect on the pulmonary hypertension through a different mechanism and possibly acting synergistically.

Iloprost (see U.S. Pat. No. 4,692,464; incorporated herein in its entirety by reference thereto) is a stable analogue of prostacyclin that is associated with a longer duration of vasodilatation (Fitscha P. et al. 1987 Adv Prostaglandin Thromboxane Leukot Res 17:450-454). When administered by aerosolization to patients with pulmonary hypertension, its pulmonary vasodilative potency was similar to that of prostacyclin, but its effects lasted for 30 to 90 minutes, as compared with only 15 minutes for the prostacyclin (Hoeper M. M. et al. 2000 J Am Coll Cardiol 35:176-182; Olschewski H. et al. 1999 Am J Respir Crit Care Med 160:600-607; Olschewski H. et al. 1996 Ann Intern Med 124:820-824; Gessler T. et al. 2001 Eur Respir J 17:14-19; Wensel R. et al. 2000 Circulation 101:2388-2392). Several open-label, uncontrolled studies of patients with severe pulmonary hypertension suggested that long-term use of aerosolized iloprost results in substantial clinical improvement (Olschewski H. et al. 1999 Am J Respir Crit Care Med 160:600-607; Olschewski H. et al. 1996 Ann Intern Med 124:820-824; Hoeper M. M. et al. 2000 N Engl J Med 342:1866-1870; Olschewski H. et al. 1998 Intensive Care Med 24:631-634.; Stricker H. et al. 1999 Schweiz Med Wochenschr 129:923-927; Olschewski H. et al. 2000 Ann Intern Med 132:435-443; Beghetti M. et al. 2001 Heart 86:E10-E10). A multi-center randomized placebo controlled study of patients with severe PAH has demonstrated improved exercise capacity in patients receiving iloprost versus those receiving placebo (Olschewski H et al 2002 NEJM 2002;345:322-9).

Endothelin Receptor Antagonists (ETRA)

There is increasing evidence that endothelin-1 has a pathogenic role in pulmonary arterial hypertension and that blockade of endothelin receptors may be beneficial. Endothelin-1 is a potent endogenous vasoconstrictor and smooth-muscle mitogen that is overexpressed in the plasma and lung tissue of patients with pulmonary arterial hypertension. There are two classes of endothelin receptors: Endothelin A, ET-A and Endothelin B, ET-B receptors, which play significantly different roles in regulating blood vessel diameter. The binding of endothelin to ET-A receptors located on smooth muscle cells causes vasoconstriction, whereas the binding of endothelin to ET-B receptors located on the vascular endothelium causes vasodilatation through the production of nitric oxide. This latter activity of the ET-B receptor is thought to be counter-regulatory and protects against excessive vasoconstriction.

Therefore, another attractive approach to treating pulmonary hypertension has been the blockade of these endothelin receptors. Two types of ETRAs have been developed: dual ETRAs, which block the receptors for both ET-A and ET-B, and selective ETRAs, which block only the ET-A receptor.

a) Dual Endothelin Receptor Antagonist

The first generation ETRAs are non-selective and block both the ET-A and ET-B receptors. Bosentan (Tracleer™) is the first FDA approved ETRA (see U.S. Pat. No. 5,292,740; incorporated herein in its entirety by reference thereto). Two placebo controlled trials of bosentan (an endothelin receptor A and B antagonist) have been conducted (Channick R. N. et al. 2001 Lancet 358:1119-1123; Rubin L. J. et al. 2002 N Engl J Med 346:896-903). The six minute walk test improved in the whole group, but the improvement was greater when the drug was used in higher doses. However, liver toxicity occurred with the higher dose.

b) Selective Endothelin Receptor Antagonist

Second generation ETRAs bind to the ET-A receptor in preference to the ET-B receptor. Currently, there are two selective ETRAs in clinical trials: sitaxsentan and ambrisentan (BSF 208075). A pure endothelin A antagonist, sitaxsentan has been used in an open pilot study. This showed an improvement in the six minute walk test and a decrease in pulmonary vascular resistance of 30% (Barst R. J. et al. 2000 Circulation 102:II-427).

A more potent endothelin compound, TBC3711 (Encysive Pharmaceuticals), entered Phase I testing in December 2001. This drug holds potential for treating chronic heart failure and essential hypertension.

There are small clinical trials of using bosentan in patients that are already on other medications for the treatment of pulmonary hypertension (Hoeper M. M. et al. 2003 in: “Pulmonary Hypertension: Clinical”, Abstr. A275, May 18, 2003; Pulmonary Hypertension Roundtable 2002, Phassociation.org/mldical/advances in PH/spring 2002). In a preferred embodiment of the present invention, the combination therapy comprises iloprost and bosentan acting in combination through distinct mechanisms of action, preferably synergistically, to treat pulmonary hypertension. In yet another preferred embodiment, iloprost is combined with sitaxentan. In yet another embodiment, iloprost is combined with ambrisentan. In yet another embodiment iloprost is aerosolized and administered in combination with bosentan, or sitaxentan, or ambrisentan. In another embodiment, iloprost is combined with TBC3711 in combination therapy of pulmonary hypertension.

Nitric Oxide Production

Endothelial production of nitric oxide is diminished with pulmonary hypertension, prompting attempts to reverse this defect either by giving continuous inhaled nitric oxide, which is effective but difficult to administer, or by increasing the substrate for nitric oxide L-arginine (Nagaya N. et al. 2001 Am JRespir Crit Care Med 163:887-891). A trial of supplementation with L-arginine is currently under way.

PDE Inhibitors

In addition to increasing the supply of nitric oxide, attempts to directly increase cyclic nucleotide second messenger levels in the smooth muscle cells have been made. Sildenafil used for erectile dysfunction blocks the enzyme phosphodiesterase type 5 present in the corpus cavemosum of the penis and also the lungs. This raises the possibility that a phosphodiesterase inhibitor, preferably a PDE type 5 inhibitor such as sildenafil, could be a relatively selective pulmonary vasodilator. There is empirical evidence supporting the inventor's selection of PDE inhibitors as a target compound in a combination therapy (see e.g., Michelakis E. et al. 2002 Circulation 105:2398-2403; Ghofrani H. et al. 2002 Lancet 360:895-900; the disclosures of which are incorporated herein in their entirety by reference).

Although aerosolized prostacyclin (PGI₂) has been suggested for selective pulmonary vasodilation as discussed above, its effect rapidly levels off after termination of nebulization. Stabilization of the second-messenger cAMP by phosphodiesterase (PDE) inhibition has been suggested as a strategy for amplification of the vasodilative response to nebulized PGI₂. Lung PDE3/4 inhibition, achieved by intravascular or transbronchial administration of subthreshold doses of specific PDE inhibitors, synergistically amplified the pulmonary vasodilatory response to inhaled PGI₂, concomitant with an improvement in ventilation-perfusion matching and a reduction in lung edema formation. The combination of nebulized PGI₂ and PDE3/4 inhibition may thus offer a new concept for selective pulmonary vasodilation, with maintenance of gas exchange in respiratory failure and pulmonary hypertension (Schermuly R. T. et al. 2000 J Pharmacol Exp Ther 292:512-20). There are some reports of small clinical studies showing that such combination therapy may be efficacious in the treatment of pulmonary hypertension (Ghofrani et al. 2002 Crit Care Med 30:2489-92; Ghofrani et al. 2003 J Am Coll Cardiol 42:158-164; Ghofrani et al. 2002 Ann Intern Med 136:515-22).

Isozyrnes of cyclic-3′,5′-nucleotide phosphodiesterase (PDE) are a critically important component of the cyclic-3′,5′-adenosine monophosphate (cAMP) protein kinase A (PKA) signaling pathway. The superfamily of PDE isozymes consists of at least nine gene families (types): PDE1 to PDE9. Some PDE families are very diverse and consist of several subtypes and numerous PDE isoform-splice variants. PDE isozymes differ in molecular structure, catalytic properties, intracellular regulation and location, and sensitivity to selective inhibitors, as well as differential expression in various cell types.

A phosphodiesterase (PDE) inhibitor is defined herein as any drug used in the treatment of pulmonary arterial hypertension that works by blocking the inactivation of cyclic AMP. There are five major subtypes of phosphodiesterase (PDE); the drugs enoximone (inhibits PDE IV) and milrinone (Primacor®) (inhibits PDE IIIc) are most commonly used medically. Other phosphodiesterase inhibitors include Aminone (Inocor®) used to improve myocardial function, pulmonary and systemic vasodilation, and sildenafil (Viagra®), tadalafil (Cialis®) and vardenafil (LEVITRA®)—selective phosphodiesterase V inhibitors.

The BusinessWire website (webbox/bw.042803/231185439.htm) reported clinical data on tadalafil, showing that 79 percent of U.S. men of diverse ethnic origin with erectile dysfunction (ED) participating in a clinical trial reported improved erections after treatment with the investigational drug, compared to 19 percent of those receiving placebo. The results of this new study conducted in the U.S. and Puerto Rico were presented today at the 98th Annual Meeting of the American Urological Association in Chicago. ED is a condition that affects an estimated 152 million men worldwide.

Tadalafil (Cialis®) is a PDE5 inhibitor developed by Lilly ICOS LLC for the treatment of erectile dysfunction. Tadalafil is available by prescription in Europe, Australia, New Zealand, and Singapore and has been approved by the U.S. FDA.

“Treatment with Cialis significantly improved erectile function, including increasing the number of successful attempts at penetration and intercourse, and the improvement of erections,” said Allen Seftel, M. D, study author and associate professor of urology at the University Hospitals of Cleveland. “I was pleased with the tolerability profile seen in these U.S. men of diverse ethnic origin, with mild to severe ED.”

In a randomized, placebo-controlled clinical study designed to evaluate the efficacy and safety of Cialis in men with mild-to-severe ED, 207 participants in the U.S. and Puerto Rico were assigned to receive either a 20 mg dose of Cialis or placebo over a 12-week period. The treatment phase was preceded by a treatment-free period of four weeks to determine baseline erectile function. Patients were advised to take the drug as needed, at the time of their choosing prior to sexual activity, and were informed that Cialis may be effective for up to 36 hours. In the study, men were advised to eat normal meals with no restrictions on fat content.

In the study, 79 percent of patients treated with Cialis reported improved erections, as determined by the Global Assessment Question, compared to 19 percent on placebo. Additional findings revealed that 77 percent of attempts at vaginal penetration, as recorded in the Sexual Encounter Profile diary, were successful in men taking Cialis, compared to 43 percent on placebo (p less than 0.001). Furthermore, men taking Cialis were able to complete 64 percent of attempts for sexual intercourse versus 23 percent of attempts for men taking placebo (p less than 0.001). Finally, men taking Cialis achieved statistically significant improvements compared to placebo for all other endpoints.

The most commonly reported (greater than or equal to 5 percent) treatment-emergent adverse effects in the study were headache, back pain, and upset stomach. The number of patients taking Cialis who discontinued the study because of adverse events was 5 percent, compared to 2 percent for placebo.

A second clinical study presented at the annual meeting of the American Urological Association was designed to evaluate the long-term safety and tolerability of Cialis in 1,173 men with ED who had previously been enrolled in Phase III clinical studies of Cialis conducted in multiple countries worldwide. These men included those who had a range of co-morbid conditions associated with ED, such as cardiovascular disease and diabetes mellitus. Data reported were from patients who had completed at least one year of treatment. All study participants initially received 10 mg of Cialis; during the assessment period, 83 percent (n=970) of these patients increased their dosage to 20 mg. Patients were advised to take the treatment as needed prior to sexual activity.

Similar to other Cialis clinical trials, the most commonly reported treatment-emergent adverse effects in the study were headache and upset stomach. Five percent of patients discontinued the study due to side effects. The discontinuation rate in this study for any individual adverse event was less than 1%.

Bayer reported on its website that LEVITRA® (vardenafil HCl) has been approved by the U.S. Food and Drug Administration (FDA) for the treatment of erectile dysfunction (ED). Levitra is now available in pharmacies nationwides.

“In clinical trials, Levitra was shown to work quickly. More importantly, Levitra was shown to improve the sexual response for the majority of men the first time they took it, and it worked consistently over time,” said Myron Murdock, M. D., Levitra investigator and nationally recognized expert in the field of male sexual dysfunction.

Bayer and GSK evaluated Levitra in an extensive clinical trial program that included more than 50 trials involving more than 5,700 men. Results from phase III clinical studies showed that Levitra:
Helped men get and keep an erection sufficient for satisfactory sexual performance
Provided first-time success and reliable improvement of erection quality for many men,
Worked in men of various ages and race and in those with co-existing medical conditions, such as diabetes, and in men who have had their prostate removed
Demonstrated a rapid response, allowing a man to initiate or respond to sexual stimulation when the time is right
Can be taken without regard to meals making it convenient for use

Levitra is a medicine that may be used up to once a day to treat erectile dysfunction (ED). Levitra is for use by prescription oniy. Men taking nitrate drugs, often used to control chest pain (also known as angina), should not take Levitra. Men who use alpha blockers, sometimes prescribed for high blood pressure or prostate symptoms, also should not take Levitra. Such combinations could cause blood pressure to drop to an unsafe level. The most commonly reported side effects are headache, flushing, and stuffy or runny nose. Men who experience an erection for more than four hours should seek immediate medical attention.

For detailed information about Levitra, see Levitra website, the disclosure of which is incorporated herein in its entirety by reference.

Calcium Channel Blockers

In accordance with one embodiment of the present invention, a prostacyclin, preferably iloprost, is administered in combination with a second agent, which is a calcium channel blockers. Calcium channel blockers, or antagonists, act by blocking the entry of calcium into muscle cells of heart and arteries so that the contraction of the heart decreases and the arteries dilate. With the dilation of the arteries, arterial pressure is reduced so that it is easier for the heart to pump blood. This also reduces the heart's oxygen requirement. Calcium channel blockers are useful for treating PPH. Due to blood pressure lowering effects, calcium channel blockers are also useful to treat high blood pressure. Because they slow the heart rate, calcium channel blockers may be used to treat rapid heart rhythms such as atrial fibrillation. Calcium channel blockers are also administered to patients after a heart attack and may be helpful in treatment of arteriosclerosis.

Calcium channel blockers which are within the scope of this invention include, but are not limited to: amlodipine (U.S. Pat. No. 4,572,909); bepridil (U.S. Pat. No. 3,962,238); clentiazem (U.S. Pat. No. 4,567,175); diltiazem (U.S. Pat. No. 3,562,257); fendiline (U.S. Pat. No. 3,262,977); gallopamil (U.S. Pat. No. 3,261,859); mibefradil (U.S. Pat. No. 4,808,605); prenylamine (U.S. Pat. No. 3,152,173); semotiadil (U.S. Pat. No. 4,786,635); terodiline (U.S. Pat. No. 3,371,014); verapamil (U.S. Pat. No. 3,261,859); aranidipine (U.S. Pat. No. 4,446,325); bamidipine (U.S. Pat. No. 4,220,649): benidipine (European Patent Application Publication No. 106,275); cilnidipine (U.S. Pat. No. 4,672,068); efonidipine (U.S. Pat. No. 4,885,284); elgodipine (U.S. Pat. No. 4,952,592); felodipine (U.S. Pat. No. 4,264,611); isradipine (U.S. Pat. No. 4,466,972); lacidipine (U.S. Pat. No. 4,801,599); lercanidipine (U.S. Pat. No. 4,705,797); manidipine (U.S. Pat. No. 4,892,875); nicardipine (U.S. Pat. No. 3,985,758); nifedipine (U.S. Pat. No. 3,485,847); nilvadipine (U.S. Pat. No. 4,338,322); nimodipine (U.S. Pat. No. 3,799,934); nisoldipine (U.S. Pat. No. 4,154,839); nitrendipine (U.S. Pat. No. 3,799,934); cinnarizine (U.S. Pat. No. 2,882,271); flunarizine (U.S. Pat. No. 3,773,939); lidoflazine (U.S. Pat. No. 3,267,104); lomerizine (U.S. Pat. No. 4,663,325); bencyclane (Hungarian Patent No. 151,865); etafenone (German Patent No. 1,265,758); and perhexiline (British Patent No. 1,025,578). The disclosures of all such patents and patent applications are incorporated herein by reference.

Preferred calcium channel blockers comprise amlodipine, diltiazem, isradipine, nicardipine, nifedipine, nimodipine, nisoldipine, nitrendipine, and verapamil, or, e.g., dependent on the specific calcium channel blockers, a pharmaceutically acceptable salt thereof.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound of formula comprises e.g., both a carboxy and an amino group.

In accordance with one preferred embodiment of the present combination therapy, iloprost is administered together with a second generation calcium antagonist, such as amlodipine. The combination may administered in a sustained release dosage form. Preferably, the combination dosage and release form is optimized for the treatment of hypertensive patients.

While a number of preferred embodiments of the invention and variations thereof have been described in detail, other modifications and methods of using the disclosed therapeutic combinations will be apparent to those of skill in the art. Accordingly, it should be understood that various applications, modifications, and substitutions may be made of equivalents without departing from the spirit of the invention or the scope of the claims. Further, it should be understood that the invention is not limited to the embodiments set forth herein for purposes of exemplification, but is to be defined only by a fair reading of the appended claims, including the full range of equivalency to which each element thereof is entitled.

All of the references cited herein are incorporated in their entirety by reference thereto.

1. A therapeutic combination for the treatment of PAH, comprising a prostacyclin and at least one additional agent, selected from the group consisting of an endothelin receptor antagonist, a PDE inhibitor, and a calcium channel blocker, wherein the prostacyclin and the at least one additional agent are provided at dosages sufficient to ameliorate at least one symptom associated with PAH.

2. The therapeutic combination of claim 1, wherein the prostacyclin is selected from the group consisting of iloprost, treprostinol, and beraprost.

3. The therapeutic combination of claim 1, wherein the endothelin receptor antagonist is selected from the group consisting of bosentan, sitaxentan, and ambrisentan.

4. The therapeutic combination of claim 1, wherein the prostacyclin is iloprost and the at least one additional agent is bosentan.

5. The therapeutic combination of claim 4, wherein the iloprost is aerosolized.

6. The therapeutic combination of claim 1, wherein the at least one additional agent comprises a PDE inhibitor selected from the group consisting of sildenafil (Viagra®), tadalafil (Cialis®) and vardenafil (LEVITRA®).

7. A method of treating PAH, comprising administering effective amounts of a therapeutic combination comprising a prostacyclin and at least one additional agent, selected from the group consisting of an endothelin receptor antagonist, a PDE inhibitor, and a calcium channel blocker.

8. The method of claim 7, wherein the prostacyclin is selected from the group consisting of iloprost, treprostinol, and beraprost.

9. The method of claim 7, wherein the endothelin receptor antagonist is selected from the group consisting of bosentan, sitaxentan, and ambrisentan.

10. The method of claim 7, wherein the prostacyclin is iloprost and the at least one additional agent is bosentan.

11. The method of claim 7, wherein the iloprost is aerosolized.

12. The method of claim 7, wherein the at least one additional agent comprises a PDE inhibitor selected from the group consisting of sildenafil (Viagra®), tadalafil (Cialis®) and vardenafil (LEVITRA®).
